(19) United States
(12) Patent Application Publication
(10) Pub. No.: US 2009/0209933 A1
(43) Pub. Date: Aug. 20, 2009

(54) WHOLE BLOOD COLLECTION KIT AND METHOD

(75) Inventors: Javier Zylberberg, Delray Beach, FL (US); Claudia Zylberberg, Delray Beach, FL (US); Ronald O. Gilcher, Oklahoma City, OK (US)

Correspondence Address: AKERMAN SENTERFITT, P.O. BOX 3188, WEST PALM BEACH, FL, 33402-3188, US

(73) Assignee: AssureImmune, LLC, Boca Raton, FL (US)

(21) Appl. No.: 12/032,124

(22) Filed: Feb. 15, 2008

(51) Int. Cl. A61J 1/10; A61M 5/32; B65D 81/38

(52) U.S. Cl. 604/408; 604/262; 604/263; 220/592.2

(57) ABSTRACT

A blood collection device includes a collection container, a flexible conduit permanently affixed at a proximal end to the collection container, and a needle permanently affixed to a distal end of the flexible conduit. The collection container, flexible conduit and needle can be provided within a package. A method for collecting blood products is also disclosed.

### BACKGROUND OF THE INVENTION

The collection of whole blood for various treatments has been and remains an important part of medicine. More recently, the collection of whole blood has gained additional importance as adult stem cells can be obtained from whole blood stocks. Stem cells are used in a number of new and promising therapies, and more therapies utilizing stem cells are being devised every year. The collection of whole blood is performed on donors, or sometimes the patient, according to procedures and with equipment that has changed little in many years. A large (usually 16 gauge) needle attached to a flexible conduit is used to withdraw the blood from the donor. The flexible conduit has a connector on a proximal end. The proximal connector on the flexible conduit is used to connect the conduit to a collection container. The collection container, or a conduit extending from the collection container, has a cooperating connector for connecting to the proximal connector to permit blood to flow from the needle into the collection container. The collection container can have one or more preservatives to preserve the collected blood and/or cells. This collection system can subject the user to exposure to blood products and the risks associated with such exposure, and can require the secure disposal of multiple pieces of equipment.

### SUMMARY OF THE INVENTION

A blood collection device includes a collection container, a flexible conduit permanently affixed at a proximal end to the collection container, and a needle permanently affixed to a distal end of the flexible conduit. The collection container, flexible conduit and needle can be provided within a package. In one embodiment, the needle is a 17 gauge ultra thin wall needle.

The collection container can include a preservative for whole blood or blood products such as stem cells. The preservative can comprise at least one selected from the group consisting of a anticoagulant, citrate phosphate, dextrose and adenine.

A needle guard can be provided for shielding the needle before use. A shipping container can be provided and can have insulating padding for protecting the collection container during shipping. A valve can be provided for preventing fluid flow from the container.

A method for collecting blood from a donor includes the steps of providing a whole blood collection device comprising a collection container, a flexible conduit permanently affixed at a proximal end to the collection container, and a needle permanently affixed to a distal end of the flexible conduit; and introducing the needle into a donor in order to cause whole blood to flow from the donor into the collection container. The collection container, flexible conduit and needle can be provided within a package, and the collection container, flexible conduit and needle are removed from the package prior to use. After blood has been collected in the collection container, a needle guard can be placed over the needle. The collection container can be placed in a shipping container and transported in the shipping container to a processing, collection, and/or storage facility.

### BRIEF DESCRIPTION OF THE DRAWINGS

There is shown in the drawings embodiments which are presently preferred, it being understood, however, that the invention can be embodied in other forms without departing from the spirit or essential attributes thereof, wherein:

FIG. 1 is a front elevation of a blood collection device and package according to the invention.

FIG. 2 is a front elevation of a blood collection device.

FIG. 3 is an exploded side elevation of a blood collection needle and flexible conduit according to the invention.

FIG. 4 is a perspective view of a blood collection kit according to the invention.

### DETAILED DESCRIPTION OF THE INVENTION

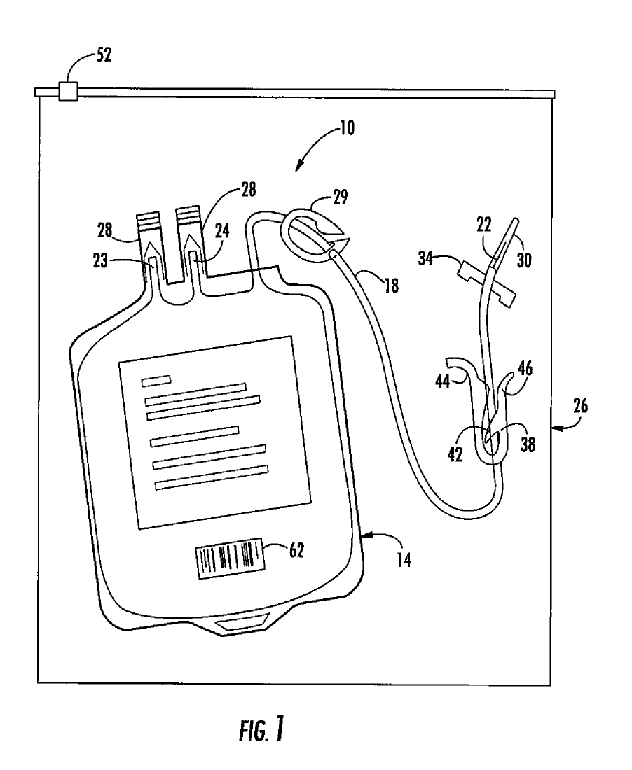

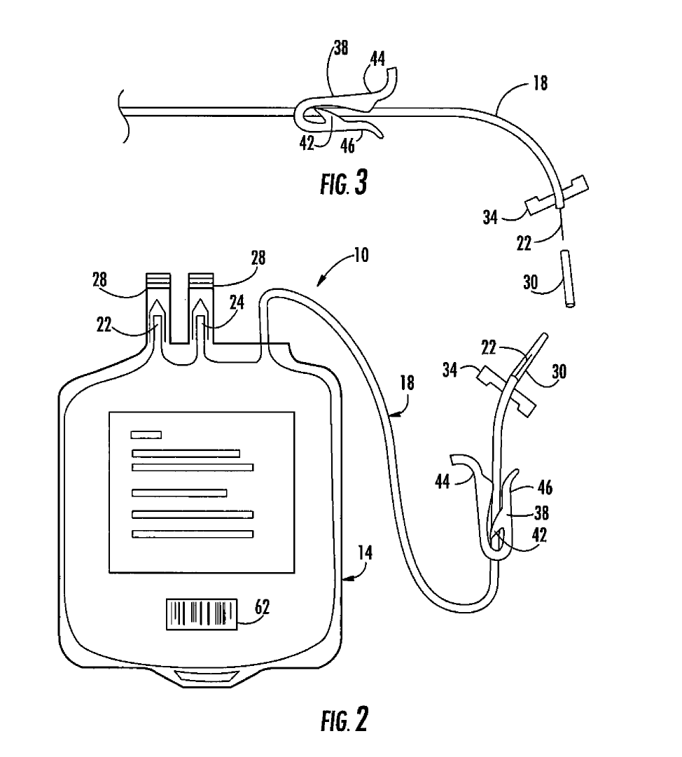

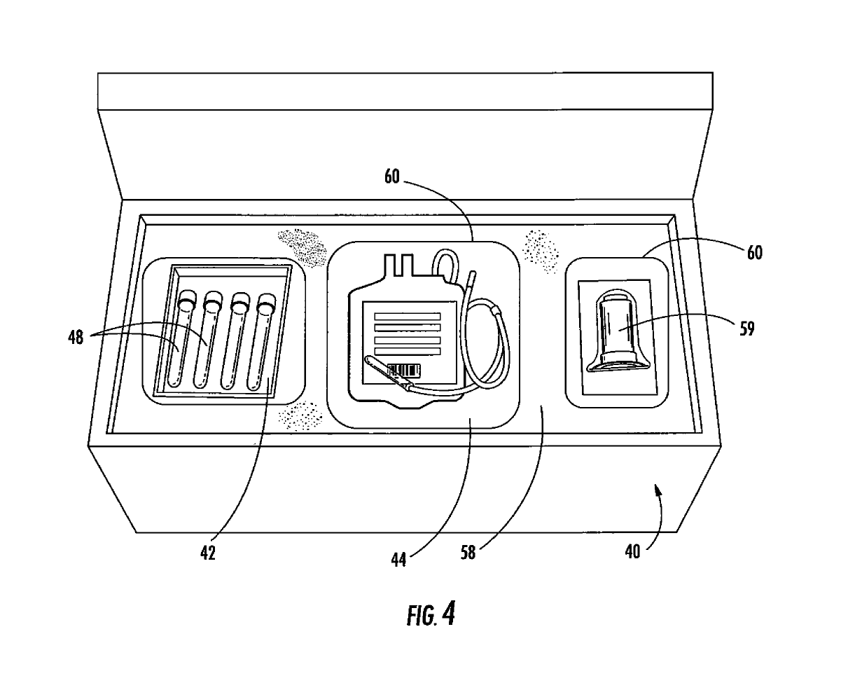

There is shown in FIGS. 1-3 a blood collection device 10. The device 10 is particularly useful for the collection of whole blood, however, it will be understood that the invention can be utilized for the collection of blood products or other biological products. The device 10 includes a collection container 14, a flexible conduit 18 permanently affixed at a proximal end to the collection container 14, and a needle 20 affixed to a distal end of the flexible conduit 18. The collection container 14, flexible conduit 18 and needle 22 can be provided within a reclosable package 26.

The collection container 14 can be of any suitable size and shape. In one embodiment, the collection container 14 is substantially rectangular, and is dimensioned to hold between about 400 ml and about 500 ml of blood or blood products. The collection container can be made of flexible plastics, generally in the form of a bag. Suitable plastics include polyvinyl chloride (PVC) and ethylene vinyl acetate (EVA). The collection container 14 can be made from a material that is transparent or translucent, so that the contents are visible or partially visible. The collection container 14 can have one or more ports such as female ports 23 and 24, which can be shielded by protective flaps 28. The flaps 28 are sealed to preserve sterility, and at the time of use are pulled apart to access the ports 23 and 24 to introduce or written materials into or from the container, such as preservatives or nutrients.

The collection container 14 can be pre-filled with one or more compositions for preserving the blood and cells that will be placed into the container 14. Such compositions can include an anticoagulant such as citrate and preservatives such as dextrose and adenine. Such solutions include citrate-phosphate-dextrose (CPD), citrate-phosphate-dextrose-adenine (CPDA-1), citrate-phosphate-double dextrose (CP2D), Adsol (AS-1), Nutricel used with CP2D anticoagulant (AS-3), or other solutions for preserving blood, blood products, and/or stem cells.

The flexible conduit 18 is preferably in the shape of a tube. The flexible conduit 18 can be of varying inside and outside diameters, and also of varying lengths. The flexible conduit 18 can be constructed from various materials such as medical grade plastics. In one embodiment the flexible conduit is about 12″ long. The conduit 18 is permanently affixed to the collection container 14. This can be accomplished by molding the collection container 14 and the conduit 18 from the same material during the molding process, such the collection container 14 and the conduit 18 form an integral piece. In another embodiment, the flexible conduit is affixed by permanently adhering the conduit 18 to the container 14. Permanently adhered as used herein means that the conduit 18 is adhered to the container 14 in such fashion that removal of the conduit 18 from the container 14 will result in damage to one or both of the container 14 and the conduit 18. The manner of affixing the conduit 18 to the container 14 can vary, and can include suitable methods such as adhesives, plastic welds, and the like.

The needle 20 is permanently affixed to the conduit 18. Permanently affixed as used herein means that the needle 20 is joined to the conduit 18 in such fashion that removal of the needle 20 from the conduit 18 will result in damage to one or both of the needle 20 and the conduit 18. The manner of affixing the needle 20 to the conduit 18 can vary, and can include suitable methods such as adhesives, plastic welds, and the like. The needle can be of varying designs and sizes. In one embodiment, the needle 20 is a 17 gauge, ultra thin wall needle which has an interior diameter the same or similar to that of a standard 16 gauge needle but because of the thin wall is more comfortable for the patient. The needle can have varying lengths, but in one embodiment has a one inch cannula. A valve such as exterior valve 29 can be utilized to prevent leakage from the container 14 through the conduit 18. Other valves or seals to prevent leakage are possible.

Various other components can be provided. A cover 30 for the needle 20 can be provided to protect the needle 20 and those handling the device 10 from needle sticks during handling and storage of the device 10. The cover 30 can be of any suitable material, and in one embodiment is in the form of a plastic tube which fits over the needle 20 and engages the needle or cooperating structure on the conduit 18 until the device is to be used, whereupon the cover 30 can be removed from the needle 20. Wings 34 can be provided on the conduit 18 to facilitate manipulation of the needle 20 and to provide a structure for taping or securing the needle 20 or conduit 18 in place on a patient or donor. A needle guard 38 can be provided to lock in place over the needle 20 after use to prevent needle sticks. A protrusion 42 on the needle guard 38 can be provided to engage the wings 34 and thereby secure flanges 44, 46 in place over the needle 20.

The package 26 can be of any suitable size and design. The package 26 can be reclosable and can have a zipper 52 or other suitable structure for this purpose. A shipping container can also be provided to protect the container 14 and its contents during shipment to a processing and/or storage facility. There is shown in FIG. 4 a kit comprising exterior container 40, and one or more interior containers 42 and 44. The interior container 42 has a number of sample vials 48. The sample vials 48 are used to collect samples that can be separately sent to various testing facilities. The interior containers 42 and 44 can serve as shipping containers, and for this purpose have interior foam or padding 58 for protecting the container 14 or vials 48 against shocks received during the shipping process, and also to act as an insulator for protecting the container 14 or vials 48 and their contents from external temperature extremes. Stem cells are preferably maintained during shipment at about 75° F.±10° F. The foam or padding 58 can be of any thickness and in one embodiment is at least about 1″ thick. The foam or padding 58 can have a cutout portions 60 for receiving the container 14, vials 48, or other contents. Other items suitable or desirable for collection such as sampling tube adapter 59 can be included in the kit. The package can be of any suitable size and shape, and in one embodiment is about 12.5″×7.5″×4.5″. The container 14 can alternatively be returned to a collection center by standard shipping services and packaging.

In practice, the device 10 can be provided as a kit within the recloseable package 26. The device 10 and package 26 can be provided in interior container 44. Collection vials 48 or the like can be provided in another interior container 42. Interior containers 42 and 44 can be provided in an exterior container 40, which is used to send the collection device to a user. The collection container 14 with the conduit 18 and needle 20 are removed from the package 26 and the needle 20 is inserted into the patient and the blood or biological material are withdrawn into the collection container 14. The valve 29 can be used to close the flexible conduit 18 against leakage and the needle guard 38 can be used for shielding the needle 20 before and after use. The collection container 14 with the conduit 18 and needle 20 can be placed into container 44, which can be used as a shipping container, and then the container 44 is sealed and transported to a collection or processing facility. A bar code 62 or other identification means can be used to identify the patient and other collection data relevant to the sample.

Although the invention has been described specifically for use in collecting blood products, the invention may also have utility in collecting other biological products from patients and donors. This invention can be embodied in other forms without departing from the spirit or essential attributes thereof and, accordingly, reference should be had to the following claims rather than the foregoing specification as indicating the scope of the invention.

1. A blood collection device, comprising:
a collection container;
a flexible conduit permanently affixed at a proximal end to the collection container; and,
a needle permanently affixed to a distal end of the flexible conduit.

2. The collection device of claim 1, further comprising a package, said collection container, flexible conduit and needle being provided within said package.

3. The blood collection device of claim 1, wherein said needle is a 17 gauge ultra thin wall needle.

4. The blood collection device of claim 1, wherein said collection container comprises a preservative for said whole blood.

5. The blood collection device of claim 1, wherein said preservative comprises at least one selected from the group consisting of a anticoagulant, citrate phosphate, dextrose and adenine.

6. The blood collection device of claim 1, further comprising a needle guard for shielding said needle before use.

7. The blood collection device of claim 1, further comprising a shipping container.

8. The blood collection device of claim 1, wherein said shipping container comprises insulating padding for protecting said container during shipping.

9. The blood collection device of claim 1, further comprising a valve for preventing fluid flow from said container.

10. A method for collecting blood from a donor, comprising the steps of:
providing a whole blood collection device comprising a collection container, a flexible conduit permanently affixed at a proximal end to the collection container, and a needle permanently affixed to a distal end of the flexible conduit; and,
introducing said needle into a donor in order to cause whole blood to flow from said donor into said collection container.

11. The method of claim 10, wherein said collection container, flexible conduit and needle are provided within a package, and removed from said package prior to use.

11. The method of claim 10 wherein, after blood has been collected in said collection container, a valve is used to prevent leakage from said collection container and a needle guard is placed over said needle.

12. The method of claim 10, wherein after blood has been collected in said collection container, said collection container is placed in a shipping container and transported in said package to a collection facility.
